(19) United States
(12) Patent Application Publication
(10) Pub. No.: US 2018/0116942 A1
(43) Pub. Date: May 3, 2018

(54) COMPOSITIONS FOR CHEMICALLY TREATED HAIR

(71) Applicant: L'OREAL, Paris (FR)

(72) Inventors: Anand MAHADESHWAR, Scotch Plains, NY (US); Kwana PATTERSON, Union, NJ (US); Barbara MITCHELL, Teaneck, NJ (US)

(21) Appl. No.: 15/339,035

(22) Filed: Oct. 31, 2016

(51) Int. Cl. A61K 8/81; A61K 8/41; A61K 8/362; A61Q 5/00; A45D 7/04

(52) U.S. Cl. CPC A61K 8/817; A61K 8/41; A61K 2800/884; A61Q 5/002; A45D 7/04; A61K 8/362

(57) ABSTRACT

The disclosure relates to compositions for treating keratinous substrates, such as the hair. The compositions comprise at least one amino acid, at least one carboxylic acid, at least one amine, and at least one cationic polymer. Methods of treating keratinous substrates with the compositions, and kits comprising the compositions, are also disclosed.

### TECHNICAL FIELD

The present disclosure relates to compositions for use with chemical treatment of the hair. The disclosure also relates to processes for treating the hair comprising applying compositions described herein to the hair.

### BACKGROUND

It is known that consumers desire to use cosmetic and personal care compositions that enhance the appearance of keratinous substrates such as hair, e.g., by changing the color, style, and/or shape of the hair and/or by imparting various properties to hair, such as shine and conditioning. Many of the known compositions and processes for enhancing the appearance of the hair involve chemical treatment of the hair.

The process of changing the color of hair, for example, can involve depositing an artificial color onto the hair which provides a different shade or color to the hair and/or lifting the color of the hair, such as lightening the color of dark hair to lighter shades. The process of lifting the color of hair, also known as lightening, generally requires the use of compositions that comprise at least one oxidizing agent.

Lightening or lifting the color of the hair is typically evaluated by the variation in tone height before and after the application of a hair color-altering composition onto hair. This variation corresponds to the degree or level of lightening or lift. The notion of “tone” is based on the classification of the natural shades, one tone separating each shade from the shade immediately following or preceding it, which is well known to hairstyling professionals. The tone heights or levels range from 1 (black) to 10 (light blond), one unit corresponding to one tone; thus, the higher the number, the lighter the shade or the greater the degree of lift.

In general, hair lightening or color lifting compositions and hair dyeing compositions possess an alkalinity such that these compositions have a pH value of above 7, typically being at pH 9 and above, and may generally require the presence of an alkalizing agent such as ammonia or an ammonia gas generating compound and/or an amine or ammonium-based compound in amounts sufficient to make such compositions alkaline. The alkalizing agent causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair.

Additionally, there are many techniques and compositions for shaping or altering the shape of hair. For example, hair care products referred to as “hair relaxers” or “hair straighteners” can relax or straighten curly or kinky hair, including wavy hair. Straightening or relaxing the curls of very curly hair may increase the manageability and ease of styling of such hair. Compositions for permanent waving the hair will impart a curl or a wave to otherwise straight hair. Different types of compositions can be applied onto hair in order to change its shape and make it more manageable, such as alkaline compositions. Hair relaxers, straighteners, perms, and/or waves may either be applied in a hair salon by a professional or in the home by the individual consumer.

While such hair dyeing, color lifting, relaxing, straightening, perming, and waving compositions can effectively alter hair color and/or shape, these chemical treatments can damage the hair. Thus, in order to prevent or repair such damage, the use of new and additional ingredients and novel combinations of ingredients are continuously sought. For example, when chemical treatments result in hair that is at an alkaline pH, the cuticles are open causing the hair to appear rough and jagged. Neutralizing rinses are often employed to reduce the pH of the hair in order to close the hair fiber and impart a smooth, soft, and shiny appearance to the hair. Thus, new and additional ingredients and novel combinations are continuously sought to reduce the pH of the hair after chemical treatment.

However, the choice of ingredients or combinations of ingredients could pose difficulties insofar as they should not be detrimental to other cosmetic attributes such as ease and uniformity of application, rheology or viscosity properties, and stability of the compositions, color deposit and target shade formation, and/or result into more disadvantages such as increased damage or a less healthy look to the hair. It would, therefore, be desirable to provide the consumer with compositions and methods that can treat the hair, e.g. relax and/or deposit color onto hair in an efficient manner, while providing other cosmetic advantages such as shine, conditioning, fiber strength, and/or a healthy appearance to the hair, but avoiding or minimizing damage to the hair.

Thus, there is a need for novel compositions and methods that can provide advantageous effects such as strengthening of the hair fiber, protecting hair fibers from damage or further damage, enhanced properties such as softness, shine, conditioning, healthy appearance, while at the same time, providing desired effects such as coloring, lightening, straightening, relaxing, and/or shaping.

### SUMMARY

The present disclosure relates to compositions and methods for application to keratinous substrates that are chemically treated. For example, the compositions and methods are suitable for treating hair that is subjected to bleaching, coloring, relaxing, straightening, perming, and/or waving processes, such as post-treatment compositions and methods.

In various exemplary and non-limiting embodiments, the compositions may be useful for preventing, minimizing, and/or repairing damage caused by chemically treating the hair. Exemplary and non-limiting embodiments of the disclosure relate to compositions comprising at least one amino acid, at least one carboxylic acid, at least one amine, at least one cationic polymer, and optionally at least one solvent.

Exemplary methods comprise applying compositions comprising at least one amino acid, at least one carboxylic acid, at least one amine, at least one cationic polymer, and optionally at least one solvent to the hair before, during, and/or after chemical treatment of the hair, such as an oxidation dyeing or lightening treatment, in order to deposit color onto the hair fiber, and/or to lift or lighten the color of the hair, while providing other cosmetic advantages such as shine, conditioning, fiber strength, and/or a healthy appearance to the hair and avoiding or minimizing damage to the hair.

Exemplary methods comprise applying a composition comprising at least one amino acid, at least one carboxylic acid, at least one amine, at least one cationic polymer, and optionally at least one solvent to the hair before, during, and/or after chemical treatment of the hair, for example a hair shaping treatment such as relaxing, straightening, or permanent waving, in order to alter the shape of the hair, while providing other cosmetic advantages such as shine, conditioning, fiber strength, and/or a healthy appearance to the hair and avoiding or minimizing damage to the hair.

The disclosure thus relates to methods of using compositions comprising at least one amino acid, at least one carboxylic acid, at least one amine, at least one cationic polymer, and optionally at least one solvent in hair coloring and/or hair lightening processes, as well as hair shaping processes such as hair relaxing, straightening, permanent waving, wherein the compositions are applied to the hair before, during, and/or after the chemical treatment processes. The disclosure further relates to methods of using compositions comprising at least one amino acid, at least one cationic polymer, and optionally at least one solvent in hair coloring and/or hair lightening processes, as well as hair shaping processes such as hair relaxing, straightening, perming and waving, wherein the compositions are applied to the hair before, during, and/or after the chemical treatment processes.

In various embodiments, the disclosure is drawn to processes for chemically treating the hair, for example by lifting or lightening the color of the hair and/or by shaping or changing the shape of the hair using relaxers, straighteners, perms and waves. In one embodiment, the methods comprise applying onto hair fibers a composition comprising at least one amino acid, at least one carboxylic acid, at least one amine, at least one cationic polymer, and optionally at least one solvent, before, during, and/or after application of the chemical treatment. Such processes may, in at least some embodiments, provide shine, conditioning, fiber strength, and/or a healthy appearance to the hair, optionally while avoiding or minimizing damage to the hair.

### DETAILED DESCRIPTION

Other than in the operating examples, or where otherwise indicated, all numbers expressing quantities of ingredients and/or reaction conditions are to be understood as being modified in all instances by the term “about” which can encompass ±10%, ±8%, ±6%, ±5%, ±4%, ±3%, ±2%, ±1%, or ±0.5%.

All numbers expressing pH values are to be understood as being modified in all instances by the term “about” which encompasses up to ±3%.

“At least one” as used herein means one or more and thus includes individual components as well as mixtures/combinations.

“Keratinous substrates” or “keratinous fibers” may be chosen from, for example, human hair. The terms “keratinous substrates” or “keratinous fibers” and “hair” or “hair fibers” may be used interchangeably herein for ease of reference only and without limitation.

“Chemical treatments” as described herein may include any chemical treatment of the keratinous substrates, such as by way of non-limiting example only, oxidation dyeing, lifting, coloring, lightening (e.g., bleaching, highlighting), relaxing, perming, waving, and/or straightening the hair.

The term “altering the color” or “color-altering” and variations thereof, as used herein, may refer to lifting or lightening the color of hair. It can also refer to dyeing or coloring hair or depositing color onto the hair. In certain instances, it refers to lifting or lightening the color of hair and depositing color onto the hair at the same time or sequentially.

The term “strength” and variations thereof, as used herein, may refer to the strength of the hair fiber with respect to the ease or difficulty of breaking a hair fiber or to the amount of effort or force needed to break the fiber when the fiber is subjected to a pulling, tugging, stretching, combing, or brushing action.

The term “protecting” as used herein may refer to the prevention, minimization or reduction of damage or further damage to hair.

“Formed from,” as used herein, means obtained from chemical reaction of, wherein “chemical reaction” includes spontaneous chemical reactions and induced chemical reactions. As used herein, the phrase “formed from” is open ended and does not limit the components of the composition to those listed, e.g., as component (i) and component (ii). Furthermore, the phrase “formed from” does not limit the order of adding components to the composition or require that the listed components (e.g., components (i) and (ii)) be added to the composition before any other components.

“Hydrocarbons,” as used herein, include alkanes, alkenes, and alkynes, wherein the alkanes comprise at least one carbon, and the alkenes and alkynes each comprise at least two carbons; further wherein the hydrocarbons may be chosen from linear hydrocarbons, branched hydrocarbons, and cyclic hydrocarbons; further wherein the hydrocarbons may optionally be substituted; and further wherein the hydrocarbons may optionally further comprise at least one heteroatom intercalated in the hydrocarbon chain.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Polymers,” as defined herein, include homopolymers and copolymers formed from at least two different types of monomers.

The term “substantially free of (a component)” as defined herein means that the compositions contain no appreciable amount of the component, for example, no more than about 1% by weight, or no more than about 0.5% by weight, or no more than about 0.3% by weight, such as no more than about 0.1% by weight, based on the weight of the composition according to embodiments of the disclosure.

The term “free” or “completely free of (a component)” as defined herein means that the compositions do not contain the component in any measurable degree by standard means.

### Compositions

It has been surprisingly and unexpectedly discovered that when keratinous substrates, e.g., hair, are subjected to a chemical treatment and treated with compositions comprising at least one amino acid, at least one carboxylic acid, at least one amine, and at least one polymeric quaternary ammonium salt, improved effects such as, for example, improved fiber strength and/or protecting and/or coloring/lightening effects can be achieved.

Without wishing to be bound by theory, it is believed that compositions according to the disclosure can reduce the pH of the hair, i.e. neutralize the hair, so that the hair fiber is closed thus resulting in improved effects such as, for example, improved fiber strength and appearance (e.g., softening, smoothing, strengthening, protecting, etc., the hair).

In various embodiments, the pH of the hair after application of the compositions may be less than about 7, such as less than about 6.5, less than about 6, less than about 5.5, or less than about 5. In various embodiments, the pH of the hair after application of the compositions may range from about 2 to about 7, such as about 3 to about 6, or about 4 to about 5.

### Amino Acids

The compositions according to various embodiments comprise at least one amino acid. According to certain embodiments, the at least one amino acid is chosen from glycine, alanine, serine, proline, or mixtures thereof.

The at least one amino acid may be present in an amount ranging from about 0.01% to about 20%, by weight, based on the total weight of the composition in which it is present. For example, the amino acid may be present in an amount ranging from about 0.1% to about 15%, or about 1% to about 12%, by weight, based on the total weight of the composition in which it is present. In one exemplary embodiment, the amino acid may be present in an amount of less than about 10%, and in another embodiment, may be present in an amount greater than about 2%, by weight, based on the total weight of the composition.

### Acids

The treatment compositions according to the disclosure comprise at least one carboxylic acid. According to various exemplary and non-limiting embodiments of the disclosure, useful carboxylic acids include any organic compound containing at least two acid functional groups and at least one carbon atom.

In certain exemplary, and non-limiting embodiments, the at least one carboxylic acid may be chosen from saturated or unsaturated, substituted or unsubstituted dicarboxylic acids, salts thereof, and mixtures thereof. By way of non-limiting example, the at least one carboxylic acid may be chosen from dicarboxylic acids of the following formula:

XO₂C—R—CO₂X′

wherein:

R is a cyclic or acyclic, saturated or unsaturated, linear or branched, C₁-C₁₀₀ hydrocarbon moiety comprising from 0 to 30 double and/or triple bonds and/or from 0 to 10 rings, and optionally interrupted by 1 to 30 heteroatoms chosen from O, N and S, and optionally substituted with 1 to 30 substituents chosen from a hydrogen atom, a hydroxyl (—OH) moiety, an amino (—NH₂) moiety, a (C₁-C₃₀)alkylamino moiety, a poly(C₁-C₃₀)alkylamino moiety, a hydroxy(C₁-C₃₀)alkylamino moiety, a polyhydroxy(C₁-C₃₀)alkylamino moiety, a C₆-C₃₀ aryl moiety, and

X and X′, independently denote a hydrogen atom, an ammonium ion, a ion of an alkali metal such as Li, Na, K, or an alkaline earth metal such as Be, Mg, Ca or an ion derived from an organic amine such as an alkylamine.

It is understood that the expression cyclic hydrocarbon moiety for the purposes of the present application is understood to mean a hydrocarbon moiety consisting of one or more rings or comprising one or more rings which are pendent or in the principle chain, it being possible for the rings to be saturated or unsaturated and to be substituted with one or more C₁-C₃₀ alkyl or alkenyl or hydroxyl or amino moieties.

By way of non-limiting example only, useful carboxylic acids may include oxalic acid, malonic acid, malic acid, glutaric acid, citraconic acid, citric acid, maleic acid, glycolic acid, succinic acid, adipic acid, tartaric acid, fumaric acid, adipic acid, sebacic acid, benzoic acid, and glyoxylic acid monohydrate, as well as combinations thereof.

The compositions according to various embodiments comprise at least one saturated or unsaturated carboxylic acid.

The at least one carboxylic acid may be present in an amount ranging from about 0.01% to about 20%, by weight, based on the total weight of the composition in which it is present. For example, the at least one carboxylic acid may be present in an amount ranging from about 0.1% to about 15%, or about 1% to about 12%, by weight, based on the total weight of the composition in which it is present. In one exemplary embodiment, the at least one carboxylic acid may be present in an amount of less than about 12%. In one exemplary embodiment, the at least one carboxylic acid may be present in an amount of greater than about 1%, and in another embodiment, may be present in an amount greater than about 10%, by weight, based on the total weight of the composition. For example, the at least one carboxylic acid may be present in an amount ranging from about 0.1% to about 5%, such as about 0.5% to about 3%, about 0.75% to about 2.5%, or about 1% to about 2%. As a further example, the at least one carboxylic acid may be present in an amount ranging from about 8% to about 15%, such as about 9% to about 12%, or about 10% to about 11%.

### Amines

The compositions according to various embodiments comprise at least one amine. In some embodiments, the amine is a monoamine. In alternative embodiments, the amine is a diamine. Exemplary amines may be chosen from, but are not limited to: ethanolamine (e.g. monoethanolamine, diethanolamine, triethanolamine); 2-aminoethanol; 3-butoxypropylamine; 3-ethoxypropylamine; tetradecylamine; 1,9-diaminononane; 4,9-dioxa-1,12-dodecanediamine; 4,7,10-trioxa-1,13-tridecanediamine; ethylenediamino; N-(2-hydroxyethyl)ethylenediamine; triethylene glycol diamine; 1,11-diamino-3,6,9-trioxaundecane; 1,3-diaminopropane; 1,4-diaminobutane; 1,5-diaminopentane; 1,6-diaminohexane; 1,7-diaminoheptane; 3-(octyloxy)propan-1-amine, 2-dimethylamino; dimethylamine; triamine bis(hexamethylene)triamine; polyoxypropylene monoamine; polyoxypropylene diamine; polyoxyethylene/polyoxypropylene monoamine; polyoxyethylene diamine, polyoxypropylene monoamine, n,n-dimethylhexylamine, trimethylammonio, 2,2-bis(aminoethoxy)propane, meso-1,4-diamino-2,3-butanediol dihydrochloride, 1,5-diamino-2-methylpentane, 1,2-diaminopropane, 1,3-diaminopentane, (3s,4s)-(−)-3,4-hexanediamine dihydrochloride, tris[2-(2-methoxyethoxy)ethyl]amine, and combinations thereof. In one exemplary embodiment, the at least one amine comprises at least one ethanolamine chosen from monoethanolamine, diethanolamine, triethanolamine, and combinations thereof.

The at least one amine may be present in an amount ranging from about 0.01% to about 20%, by weight, based on the total weight of the composition in which it is present. For example, the amine may be present in an amount ranging from about 0.1% to about 15%, about 0.2% to about 10%, or about 0.5% to about 8% by weight, based on the total weight of the composition in which it is present. In one exemplary embodiment, the amine may be present in an amount of less than about 10%, such as less than about 6%, or less than about 1% by weight, and in another embodiment, may be present in an amount greater than about 0.5%, such as greater than about 1%, or greater than about 5% by weight, based on the total weight of the composition.

### Cationic Polymer

The compositions according to various embodiments comprise at least one cationic polymer. As used herein, a “cationic polymer” is any polymer containing cationic groups and/or groups which can be ionized to cationic groups. Useful cationic polymers can include polyamine, polyaminoamide and quaternary polyammonium polymers, for example.

Exemplary and non-limiting polyamine, polyaminoamide and quaternary polyammonium polymers that can be used in the composition of the present invention are described in French patents FR 2 505 348 and FR 2 542 997. These polymers include the following:

(1) homopolymers or copolymers derived from esters or amides of acrylic or methacrylic acid;

(2) cationic cellulose derivatives such as:
(a) the cellulose ether derivatives containing quaternary ammonium groups that are described, for example, in French patent FR 1 492 597;
(b) the copolymers of cellulose or cellulose derivatives grafted with a water-soluble quaternary ammonium monomer, which are described, for example, in U.S. Pat. No. 4,131,576, such as hydroxyalkylcelluloses, for instance hydroxymethyl-, hydroxyethyl- or hydroxypropyl celluloses grafted with a methacryloylethyltrimethyl-ammonium, methacrylamidopropyltrimethyl-ammonium or dimethyldiallylammonium salt. A non-limiting example that may be mentioned is polyquaternium 10;

(3) other cationic polysaccharides such as those described, for example, in U.S. Pat. No. 3,589,578 and U.S. Pat. No. 4,031,307, such as guar gums containing cationic trialkyl-ammonium groups;

(4) polymers composed of piperazinyl units and divalent alkylene or hydroxyalkylene groups having straight or branched chains which are optionally interrupted by oxygen, sulphur or nitrogen atoms or by aromatic or heterocyclic rings, and also the oxidation and/or quaternization products of these polymers. Polymers of this kind are described, for example, in French patents FR 2 162 025 and FR 2 280 361;

(5) polyaminoamides which are soluble in water, such as those described in French patents FR 2 252 840 and FR 2 368 508;

(6) polyaminoamide derivatives, for example, the adipic acid/dialkylaminohydroxy-alkyldialkylenetriamine polymers in which the alkyl group contains 1 to 4 carbon atoms and is preferably a methyl, ethyl or propyl group, and the alkylene group contains 1 to 4 carbon atoms and is preferably the ethylene group. Polymers of this kind are described, for example, in French patent FR 1 583 363;

(7) polymers obtained by reacting a polyalkylene-poly-amine containing two primary amine groups and at least one secondary amine group with a dicarboxylic acid selected from diglycolic acid and saturated aliphatic dicarboxylic acids having 3 to 8 carbon atoms. The molar ratio of the polyalkylene-polyamine to the dicarboxylic acid is between 0.8:1 and 1.4:1, and the resulting polyaminoamide is reacted with epichlorohydrin in a molar ratio of epichlorohydrin to the secondary amine group of the polyaminoamide of between 0.5:1 and 1.8:1. Polymers of this kind are described, for example, in U.S. Pat. No. 3,227,615 and U.S. Pat. No. 2,961,347;

(8) alkyldiallylamine or dialkyldiallylammonium cyclopolymers such as the homopolymer of dimethyl-diallylammonium chloride, and the copolymers of diallyldimethylammonium chloride and acrylamide;

(9) quaternary diammonium polymers having a number-average molecular mass of generally between 1000 and 100 000, such as those described, for example, in French patents FR 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020; an example that may be mentioned is hexadimethrine chloride (INCI name), sold by Chimex under the name Mexomere PO;

(10) quaternary polyammonium polymers such as those described, for example, in patent application EP-A-122 324;

(11) quaternary vinylpyrrolidone and vinylimidazole polymers such as, for example, the products sold under the names Luviquat® FC 905, FC 550 and FC 370 by BASF;

(12) polyamines such as Polyquart® H sold by Henkel, listed under the name “POLYETHYLENE GLYCOL (15) TALLOW POLYAMINE” in the CTFA dictionary; and

(13) crosslinked polymers of methacryloyloxyalkyl(C₁-C₄) trialkyl(C₁-C₄) ammonium salts, such as those sold under the name Salcare® SC 92, Salcare® SC 95 and Salcare® SC 96 by Allied Colloids.

Other cationic polymers which can be used in the context of the invention are cationic proteins or hydrolysates of cationic proteins, polyalkyleneimines, for example polyethyleneimines, polymers containing vinylpyridine or vinylpyridinium units, condensates of polyamines and epichlorohydrin, quaternary polyureylenes and chitin derivatives.

According to one embodiment, the cationic polymer is chosen from polymeric quaternary ammonium salts. As non-limiting examples, the cationic polymer is chosen from homopolymers of diallyl dimethyl ammonium chloride, such as polyquatemium-6, available from Lubrizol as Merquat™ 100 Polymer. Mixtures of cationic polymers may also be chosen.

The cationic polymer or polymers, when present, may be present in a total amount of up to about 20%, such as from about 0.001% to about 10%, from about 0.01% to about 5%, or from about 0.1% to about 3%, by weight, relative to the total weight of the composition.

### Solvent

The compositions may optionally further comprise solvents. In various exemplary and non-limiting embodiments, the solvent is chosen from cosmetically acceptable solvents. The cosmetically acceptable solvents may be chosen from water, at least one cosmetically acceptable organic solvent, and mixtures thereof.

The organic solvents may be volatile or non-volatile compounds. As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents are ethylene glycol, propylene glycol, butylene glycol, hexylene glycol, propane diol, and glycerin.

The cosmetically acceptable solvent may comprise an amount ranging up to about 90%, such as up to about 85%, up to about 80%, up to about 75%, up to about 70%, up to about 65%, or up to about 60%, for example from about 5% to about 85% by weight, from about 10% to about 80% by weight, from about 15% to about 75% by weight, or from about 20% to about 70% by weight, based on the total weight of the composition.

In at least one embodiment, the compositions according to the disclosure comprise at least one amino acid, at least one cationic polymer, and optionally at least one solvent. It may be desirable, although not required, to apply such compositions to the hair before, at substantially the same time as, or after applying to the hair a separate composition comprising at least one carboxylic acid, at least one amine, and optionally at least one solvent.

In one exemplary and non-limiting embodiment, a composition according to the disclosure comprises at least one amino acid chosen from glycine, at least one carboxylic acid chosen from maleic acid and salts thereof, at least one amine chosen from monoethanolamine, at least one cationic polymer chosen from homopolymers of diallyl dimethyl ammonium chloride (polyquaternium-6), and optionally at least one solvent chosen from water. In a further exemplary and non-limiting embodiment, a composition according to the disclosure comprises at least one amino acid chosen from glycine, at least one cationic polymer chosen from homopolymers of diallyl dimethyl ammonium chloride (polyquaternium-6), and optionally at least one solvent chosen from water.

### Chemical Treatments

As described above, it may be advantageous to use the compositions before, during, and/or after chemical treatment of the hair. In various embodiments, the above-described compositions may be formulated as pre- or post-treatment compositions that may be applied to the hair before or after chemical treatment such as coloring, bleaching, permanent waving, relaxing, and the like. In further embodiments, the compositions described herein may optionally further comprise chemical treatment components, for example at least one lightening or coloring compound, at least one agent for relaxing, straightening, or permanent waving the hair, at least one neutralizing agent, at least one nonionic surfactant, and/or at least one auxiliary agent suitable for use in cosmetic compositions, such as chelating agents and strengthening agents. In yet further embodiments, the compositions described herein may be incorporated into chemical treatment compositions. Thus, it is to be understood that the compositions described herein may be applied to the hair before or after chemical treatment compositions, for example sequentially, or may be applied to the hair simultaneously or as part of (e.g. mixed with) the chemical treatment composition, and the following description is intended to be interpreted as such, without limitation.

By way of non-limiting example, the above-described compositions may optionally be mixed with, or applied to the hair before or after, a composition comprising hair shaping agents such as permanent waving, relaxing, or straightening agents.

In yet a further non-limiting example, the above-described compositions may optionally be mixed with, or applied to the hair before or after, an oxidizing composition containing at least oxidizing agent selected from peroxides, urea peroxide, alkali metal bromates, ferricyanides, peroxygenated salts, perborates, percarbonates, laccases, peroxidases, redox enzymes, and mixtures thereof, and a cosmetically acceptable solvent selected from water and a water/organic solvent mixture. For example, hydrogen peroxide may be used. The resulting composition and the oxidizing composition may be useful for lifting or lightening the color of the hair. When the cosmetic composition additionally contains a colorant compound, the resulting composition may be useful for depositing color onto hair.

### Shaping Agents

As described herein, in various exemplary and non-limiting embodiments, chemical treatment compositions may optionally be mixed with, or applied to the hair before or after, at least one hair shaping agent or composition comprising at least one hair shaping agent, such as, for example, permanent waving, relaxing, or straightening agents. Such hair shaping agents may optionally be chosen from inorganic hydroxides or organic hydroxides, for example sodium hydroxide, potassium hydroxide, lithium hydroxide, calcium hydroxide, or guanidine hydroxide, or may be chosen from organic amines and other non-hydroxide compounds. In various embodiments, the hair relaxing agents may be chosen from thiol compounds such as cysteine, cysteamine, N-substituted cysteamines, alkyl substituted mercaptoacetamides, dimercaptoadipic acid, thioglycerol, thiolactic acid, thioglycolic acid or its salts, (e.g., a thioglycolate), monothioglycolic acid esters such as diol esters of thioglycolic acid, glyceryl monothioglycolate, thiocholine or its salts, amino thiols, and thiols attached to low molecular weight polymers, guanidine, sulfites such as sodium hyposulfite, and bisulfites such as ammonium or sodium bisulfate. Derivatives of the aforementioned compounds may also be chosen.

By way of example only, shaping compositions such as those for relaxing or straightening the hair comprising (A) a powder composition comprising at least one alkaline material comprising at least one hydroxide-containing compound selected from alkali metal hydroxides, alkaline-earth metal hydroxides, and mixtures thereof; at least one carbonate; at least one starch; at least one silica material; at least one liquid fatty substance; at least one acrylic polymer; and at least one wax; and (B) an aqueous composition containing a cosmetically acceptable solvent chosen from water and a water/organic solvent mixture, as described in U.S. Publication No. 2015/0283041, may be chosen.

In some embodiments, the at least one hydroxide-containing compound is selected from sodium hydroxide, potassium hydroxide, lithium hydroxide, calcium hydroxide, magnesium hydroxide, barium hydroxide, strontium hydroxide, and mixtures thereof.

In other embodiments, the at least one hydroxide-containing compound is selected from sodium hydroxide, potassium hydroxide, lithium hydroxide, calcium hydroxide, and mixtures thereof.

According to at least certain embodiments, the at least one hydroxide-containing compound is calcium hydroxide.

In other preferred embodiments, the calcium hydroxide is the sole hydroxide-containing compound comprising the alkaline material.

In other embodiments, the at least one hydroxide-containing compound is not sodium hydroxide or potassium hydroxide.

The at least one alkaline material comprising at least one hydroxide-containing compound may be employed in an amount ranging from about 8 to about 30% by weight, from about 10 to about 25% by weight, or, from about 15 to about 25% by weight, with all weights of the alkaline material being the weight of the active material and based on the total weight of the chemical treatment composition, including all ranges and subranges therebetween.

In certain embodiments, the at least one alkaline material comprising at least one hydroxide-containing compound is employed in an amount of about 25%, or about 23%, or about 20%, by weight, with all weights of the alkaline material being the weight of the active material and based on the total weight of the chemical treatment composition.

In various exemplary and non-limiting embodiments, chemical treatment compositions comprise at least one carbonate compound selected from lithium carbonate, sodium carbonate, potassium carbonate, guanidine carbonate, and mixtures thereof.

According to one embodiment, the at least one carbonate compound is carbonate compound is guanidine carbonate.

The at least one carbonate compound is employed in the compositions of the present invention in an amount ranging from about 8 to about 30% by weight, from about 8 to about 20% by weight, from about 10 to about 20% by weight, based on the total weight of the chemical treatment composition, including all ranges and subranges therebetween.

In certain embodiments, the at least one carbonate compound is employed in an amount of about 8%, or about 10%, or about 15%, by weight, based on the total weight of the chemical treatment composition.

It is to be understood, however, that any agent or composition for altering the shape of the hair is intended to be within the scope of the disclosure.

### Colorants

As described herein, in various exemplary and non-limiting embodiments, chemical treatment compositions may optionally be mixed with, or applied to the hair before or after, at least one colorant compound or composition comprising at least one colorant compound. The at least one colorant compound may be chosen from, for example, oxidative dye precursors, direct dyes, pigments, and mixtures thereof.

The oxidation dyes are generally chosen from one or more oxidation bases optionally combined with one or more couplers.

By way of example, the oxidation bases may be chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, may be chosen.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases can include the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-□-hydroxyethoxy-3-amino-pyrazolo[1,5-a]pyridine; 2-(4-diméthylpyperazinium-1-yl)-3-amino-pyrazolo[1,5-a]pyridine; and the addition salts thereof.

By way of example, oxidation bases can be selected from 3-aminopyrazolo-[1,5-a]-pyridines, optionally substituted on carbon atom 2 by:

(a) one (di)(C₁-C₆)(alkyl)amino group wherein said alkyl group can be substituted by at least one hydroxy, amino, imidazolium group;

(b) one heterocycloalkyl group containing from 5 to 7 members chain, and from 1 to 3 heteroatomes, potentially cationic, potentially substituted by one or more (C₁-C₆)alkyl, such as di(C₁-C₄)alkylpipérazinium; or

(c) one (C₁-C₆)alkoxy potentially substituted by one or more hydroxy groups such as α-hydroxyalkoxy, and the addition salts thereof.

Among the pyrimidine derivatives that may be mentioned are the compounds described, for example, in the patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are the compounds described in the patents DE 3843892, DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazino-pyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof. 4,5-Diamino-1-(β-methoxyethyl)pyrazole may also be used.

A 4,5-diaminopyrazole may be used, for example 4,5-diamino-1-(β-hydroxyethyl) pyrazole and/or a salt thereof.

Pyrazole derivatives that may also be mentioned include diamino-N,N-dihydropyrazolopyrazolones such as those described in patent application FR-A-2 886 136, for example the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

2,3-Diamino-6,7-dihydro-1H,5H-pyrazolo[1,2-a]pyrazol-1-one and/or a salt thereof may be used.

4,5-Diamino-1-(β-hydroxyethyl)pyrazole and/or 2,3-diamino-6,7-dihydro-1H,5H-pyrazolo[1,2-a]pyrazol-1-one and/or a salt thereof may be used as heterocyclic bases.

Compositions according to embodiments of the disclosure may optionally further comprise one or more couplers advantageously chosen from those conventionally used in the dyeing or coloring of keratinous substrates.

Among these couplers, mention may be made of meta-phenylenediamines, meta-aminophenols, meta-diphenols, naphthalene-based couplers and heterocyclic couplers, and also the addition salts thereof.

Mention may be made, for example, of 2-methyl-5-aminophenol, 5-N-(ß-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methylbenzene, 4-chloro-1,3-dihydroxybenzene, 2,4-diamino-1-(ß-hydroxyethyloxy)benzene, 2-amino-4-(ß-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-ß-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzo-morpholine, 3,5-diamino-2,6-dimethoxypyridine, 1-N-(ß-hydroxyethyl)amino-3,4-methylenedioxybenzene, 2,6-bis(ß-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methyl-pyrazolo[1,5-a]benzimidazole, the addition salts thereof with an acid, and mixtures thereof.

In general, the addition salts of the oxidation bases and couplers that may be used may be selected from the addition salts with an acid such as the hydrochlorides, hydrobromides, sulfates, citrates, succinates, tartrates, lactates, tosylates, benzenesulfonates, phosphates and acetates.

The oxidation base(s) may be present in an amount ranging from about 0.001% to 10% by weight, such as from about 0.005% to 5% by weight, relative to the total weight of the composition in which it is present.

The coupler(s), if they are present, may be present in an amount ranging from about 0.001% to 10% by weight, such as from about 0.005% to 5% by weight, relative to the total weight of the composition in which it is present.

One or more synthetic or natural direct dyes may be used, for example chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

In various embodiments, direct dyes are chosen from cationic direct dyes. Mention may be made of the hydrazono cationic dyes of formulas (Va) and (V′a), the azo cationic dyes (VIa) and (VI′a) and the diazo cationic dyes (VIIa) below:

Het⁺-C(R^{a})═N—N(R^{b})—Ar,An⁻ (Va)

Het⁺-N(R^{a})—N═C(R^{b})—Ar,An⁻ (V′a)

Het⁺-N═N—Ar,An⁻ (VIa)

Ar⁺—N═N—Ar″,An⁻ (VI′a) and

Het⁺-N═N—Ar′—N═N—Ar,An⁻ (VIIa)

wherein in formulae (Va), (V′a), (VIa), (VI′a) and (VIIa):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ represents an aryl radical, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy;
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group; or alternatively the substituent R^{a} with a substituent of Het⁺ and/or R^{b} with a substituent of Ar and/or R^{a} with R^{b} form, together with the atoms that bear them, a (hetero)cycloalkyl; wherein optionally, R^{a} and R^{b} represent a hydrogen atom or a group (C₁-C₄)alkyl, optionally substituted with a hydroxyl group; and
An⁻ represents an anionic counter-ion such as mesylate or halide.

For example, mention may be made of the azo and hydrazono cationic dyes bearing an endocyclic cationic charge of formulae (Va), (V′a) and (VIa) as defined previously. More particularly those of formulae (Va), (V′a) and (VIa) derived from the dyes described in patent applications WO 95/15144, WO 95/01772 and EP-714954.

Optionally, the cationic part may be derived from the following derivatives:

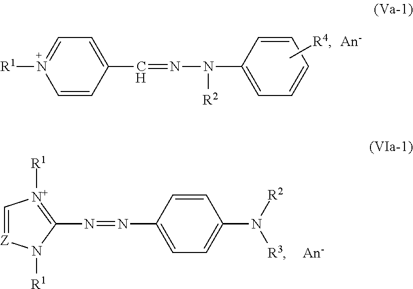

wherein in formulae (Va-1) and (VIa-1):
R¹ represents a (C₁-C₄) alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group;
Z represents a CH group or a nitrogen atom, preferentially CH; and
An⁻ represents an anionic counter-ion such as mesylate or halide.

In various embodiments, the dye of formulae (Va-1) and (VIa-1) may be chosen from Basic Red 51, Basic Yellow 87, and Basic Orange 31 or derivatives thereof:

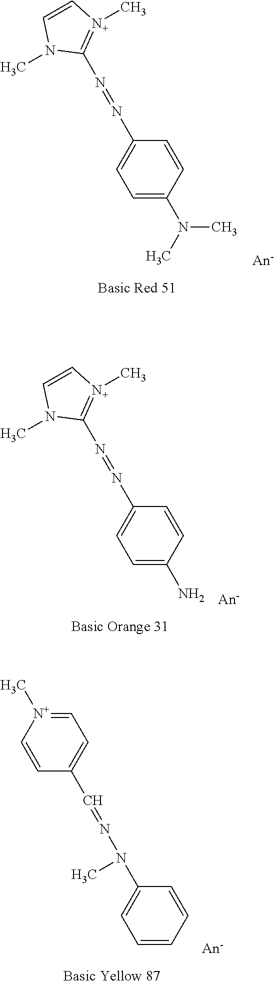

Among the natural direct dyes, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used.

When they are present, the one or more direct dyes more particularly represent from about 0.001% to 10% by weight, such as from about 0.005% to 5% by weight, of the total weight of the composition in which it is present.

### Nonionic Surfactants

As described herein, in various exemplary and non-limiting embodiments, chemical treatment compositions may optionally be mixed with, or applied to the hair before or after, at least one nonionic surfactant or composition comprising at least one nonionic surfactant. In general, nonionic surfactants having a Hydrophilic-Lipophilic Balance (“HLB”) of from 8 to 20 may be used. Non-limiting examples of nonionic surfactants useful in the compositions are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C₁₂-C₅₀ range, preferably in the C₁₆-C₄₀ range, more preferably in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Uniqema, Wilmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example Brij® 72 (i.e., Steareth-2) and Brij® 76 (i.e., Steareth-10).

Also useful herein as nonionic surfactants are alkyl glycosides, which are the condensation products of long chain alcohols, e.g. C₈-C₃₀ alcohols, with sugar or starch polymers. These compounds can be represented by the formula (S)ₙ—O—R, wherein S is a sugar moiety such as glucose, fructose, mannose, galactose, and the like; n is an integer of from about 1 to about 1000; and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. For example, nonionic surfactants can include sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. According to certain embodiments, sorbitan monoisostearate and sorbitan sesquioleate can be used as emulsifiers.

Also suitable for use herein are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being the preferred. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to about 20).

The nonionic surfactant(s) for use in the compositions can be different than the above-described fatty substance(s) employed in said compositions.

The nonionic surfactants are those formed from a fatty alcohol, a fatty acid, or a glyceride with a C₄ to C₃₆ carbon chain, a C₁₂ to C₁₈ carbon chain, a C₁₆ to C₁₈ carbon chain, derivatized to yield an HLB of at least 8. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 8. The nonionic surfactants can contain ethoxylate in a molar content of from 10-25, or from 10-20 moles.

The nonionic surfactant, if present, may be present in an amount ranging from about 0.1% to about 30% by weight, such as from about 0.5% to 20% by weight, from about 1% to about 10% by weight, or from about 1% to about 5% by weight, based on the total weight of the composition in which it is present.

### Oxidizing Agents

As described herein, in various exemplary and non-limiting embodiments, chemical treatment compositions may optionally be mixed with, or applied to the hair before or after, at least one oxidizing agent or composition comprising at least one oxidizing agent. The at least one oxidizing agent may be chosen from, for example, peroxides, persulfates, perborates percarbonates, alkali metal bromates, ferricyanides, peroxygenated salts, or a mixture thereof. Oxidizing agents that may also be used include at least one redox enzyme such as laccases, peroxidases, and 2-electron oxidoreductases, such as uricase, where appropriate in the presence of their respective donor or co-factor. By way of example, the oxidizing agent may be hydrogen peroxide. Oxygen in the air may also be employed as an oxidizing agent.

In one embodiment, the oxidizing agent can be hydrogen peroxide present in an aqueous solution whose titre may range from 1 to 40 volumes, such as from 5 to 40 volumes, or from 5 to 20 volumes.

In another embodiment, the oxidizing agent can be a persulfate and/or a monopersulfate such as, for example, potassium persulfate, sodium persulfate, ammonium persulfate, as well as mixtures thereof. In one embodiment, the oxidizing agents in the present disclosure are selected from hydrogen peroxide, potassium persulfate, sodium persulfate, and mixtures thereof.

In general, the oxidizing agent will be present in an amount ranging from about 0.05 to about 50% by weight, such as from about 0.1% to about 30% by weight, from about 0.1% to about 20% by weight, or from about 1% to about 10% by weight, based on the total weight of the oxidizing composition or solution in which it is present.

In one embodiment, the oxidizing composition is aqueous or is in the form of an emulsion.

In another embodiment, the oxidizing composition is substantially anhydrous.

The term “substantially anhydrous” means that the oxidizing composition is either completely free of water or contains no appreciable amount of water, for example, no more than 5% by weight, or no more than 2% by weight, or no more than 1% by weight, based on the weight of the oxidizing composition. It should be noted that this refers for example to bound water, such as the water of crystallization of the salts or traces of water absorbed by the raw materials used in the preparation of the compositions according to embodiments of the disclosure.

The oxidizing composition can contain at least one solvent, chosen from water, organic solvents, and mixtures thereof.

When the oxidizing composition is substantially anhydrous, the oxidizing composition may comprise at least one solvent chosen from organic solvents.

Suitable organic solvents for use in the oxidizing composition include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The organic solvents for use in the present invention can be volatile or non-volatile compounds.

The organic solvent may, for example, be present in an amount ranging from about 0.5% to about 70% by weight, such as from about 2% to about 60% by weight, preferably from about 5 to about 50% by weight, relative to the total weight of the oxidizing composition in which it is present.

The oxidizing composition may be in the form of a powder, gel, liquid, foam, lotion, cream, mousse, and emulsion.

The oxidizing composition can also be called a developer composition.

The oxidizing composition of the present invention may also contain at least one fatty substance as described above. Thus, the total amount of fatty substances in the combination or mixture of the cosmetic and oxidizing compositions of the present invention may range from about 10% to about 80% by weight, or such as from about 20% to about 60% by weight, or such as from about 20% to about 40% by weight, or such as from about 20% to about 30% by weight, based on the total weight of the composition in which it is present.

The pH of the oxidizing composition can range from about 2 to about 12, such as from about 6 to about 11, and it may be adjusted to the desired value using acidifying/alkalizing agents that are well known in the art. In certain embodiments, the pH of the oxidizing composition is below 7.

The pH of the composition resulting from mixing together the composition and the oxidizing composition may range from about 2 to about 7, such as from about 3 to about 6.9, or from about 4 to about 6.9, or from about 4 to about 6.85, or from about 5 to about 6.8.

According to at least one embodiment, the compositions and the oxidizing composition are free or substantially free of ammonia.

### Auxiliary Agents

As described herein, in various exemplary and non-limiting embodiments at least one auxiliary agent may also be present in the compositions described herein and/or in the chemical treatment compositions. The auxiliary agent may include, but are not limited to, thickening agents and rheology modifying agents, chelating agents, cationic polymers, film forming compounds, humectants and moisturizing agents, emulsifying agents other than those that fall under the above-described fatty substances, fillers, structuring agents, propellants, anionic surfactants, cationic surfactants, amphoteric surfactants, shine agents, conditioning agents, shine agents, and strengthening agents. For example, the at least one auxiliary agent may be chosen from but not limited to, Polyquaternium-34, SAMSON Supplied by Ashland, N-Hance 4572 supplied by Ashland, N-Dur-Hance A1000 supplied by Ashland, Merquat 100 supplied by Lubrizol, Merquat 2003 supplied by Lubrizol, Belsil ADM 8301 E supplied by Wacker, (ShineE503713) supplied by Wacker, Filoxane, cerafill, wheat amino acid proteins, Glycerin, ceramide, threonine.

Thickening agents and rheology modifying polymers may be chosen from polymeric thickeners and non-polymeric thickeners. The polymeric thickener can be chosen from ionic or non-ionic, associative or non-associative polymers. Exemplary polymeric thickeners include various native gums. Representative non-polymeric thickening agents include oxyethylenated molecules and especially ethoxylated alkyl or acyl derivatives of polyols. These polymers can optionally be modified physically or chemically.

If present, the at least one auxiliary agent may be present in an amount up to about 25%, such as up to about 20%, up to about 15%, or up to about 10% by weight, such as from about 0.1% to about 10% by weight, from about 0.5% to about 5%, or about 1 to about 3% by weight, based on the total weight of the composition in which it is present.

The compositions and/or chemical treatment compositions according to embodiments of the disclosure can also comprise at least one cosmetically acceptable additive used conventionally in compositions for application onto hair.

“Additive” means a substance that is added, different from the compounds already mentioned.

As examples of cosmetically acceptable additives that can be used, non-limiting mentions can be made of antioxidants or reducing agents, penetrating agents, sequestering agents, perfumes, buffers, dispersants, ceramides, sunscreen agents, preservatives, opacifiers, and antistatic agents.

The compositions and/or chemical treatment compositions of the present disclosure can be in various forms, such as in the form of liquids, creams, liquid-gels, liquid-creams, gels, lotions or pastes.

### Methods

Exemplary methods for treating the hair may comprise applying a cosmetic composition comprising at least one amino acid, at least one carboxylic acid, at least one amine, and at least one cationic polymer to the hair before, during, and/or after chemical treatment of the hair, in order to provide one or more of shine, conditioning, fiber strength, protection, and/or a healthy appearance to the hair, while avoiding, minimizing, and/or repairing damage to the hair.

According to various embodiments, compositions according to the disclosure may be applied to the hair “before” chemical treatment of the hair, with or without shampooing and/or rinsing in between, such as less than one minute before, up to about 5 minutes before, up to about 10 minutes before, up to about 20 minutes before, up to about 30 minutes before, up to about 1 hour before, up to about 2 hours before, up to about 6 hours before, up to about 12 hours before, up to about 24 hours before, up to about 48 hours before, up to about 72 hours before, or up to about 1 week before, for example. For example, a composition according to the disclosure comprising at least one amino acid; at least one carboxylic acid; at least one amine; and at least one cationic polymer may be applied as a separate step before chemical treatment of the hair is carried out.

According to further embodiments, compositions according to the disclosure may be applied to the hair “after” chemical treatment of the hair, with or without shampooing and/or rinsing in between, such as less than one minute after, up to about 5 minutes after, up to about 10 minutes after, up to about 20 minutes after, up to about 30 minutes after, up to about 1 hour after, up to about 2 hours after, up to about 6 hours after, up to about 12 hours after, up to about 24 hours after, up to about 48 hours after, up to about 72 hours after, or up to about 1 week after, for example. For example, a composition according to the disclosure comprising at least one amino acid; at least one carboxylic acid; at least one amine; and at least one cationic polymer may be applied as a separate step after chemical treatment of the hair is carried out.

According to yet further embodiments, compositions according to the disclosure may be applied to the hair “during” chemical treatment of the hair (e.g. at approximately the same time as), for example by applying the compositions as a part of the chemical treatment of the hair (e.g. the composition is incorporated into a chemical treatment, such as into a hair dyeing or hair relaxing composition, or the chemical treatment is incorporated into the composition, such as a hair relaxing agent being mixed into the composition), or for example where the composition is separate from the chemical treatment composition but is applied substantially at the same time as the composition such that both are on the hair at the same time (e.g. effecting an in-situ mixing on the hair), with or without shampooing or rinsing in between.

One exemplary and non-limiting method according to the disclosure comprises applying a composition comprising at least one amino acid chosen from glycine, at least one carboxylic acid chosen from maleic acid and salts thereof, at least one amine chosen from monoethanolamine, at least one cationic polymer chosen from homopolymers of diallyl dimethyl ammonium chloride (polyquaternium-6), and optionally at least one solvent chosen from water, to the hair before, during, and/or after chemical treatment of the hair, such as before, during, and/or after a bleaching process, a coloring process, a relaxing process, a permanent waving process, or a straightening process.

In further embodiments of the disclosure, methods according to the disclosure comprise applying a composition comprising at least one amino acid, at least one cationic polymer, and optionally at least one solvent to the hair. In various embodiments, methods according to the disclosure comprise applying a first composition comprising at least one amino acid, at least one cationic polymer, and optionally at least one solvent to the hair before, at substantially the same time as, or after applying to the hair a second composition comprising at least one carboxylic acid, at least one amine, and optionally at least one solvent.

In exemplary methods where a first composition comprising at least one amino acid, at least one cationic polymer, and optionally at least one solvent is applied to the hair before or after a second composition comprising at least one carboxylic acid, at least one amine, and optionally at least one solvent is applied to the hair, the hair may optionally be rinsed and/or washed in between application of the first and second compositions, and there may be an optional leave-on period of time between application of the first and second compositions.

In exemplary methods where a first composition comprising at least one amino acid, at least one cationic polymer, and optionally at least one solvent is applied to the hair at substantially the same time as a second composition comprising at least one carboxylic acid, at least one amine, and optionally at least one solvent is applied to the hair, the two compositions may be mixed before application to the hair, or may be separately applied to the hair at substantially the same time (e.g. effecting an in-situ mixing on the hair).

A further exemplary and non-limiting method according to the disclosure comprises applying a composition comprising at least one amino acid chosen from glycine, at least one cationic polymer chosen from homopolymers of diallyl dimethyl ammonium chloride (polyquaternium-6), and optionally at least one solvent chosen from water, to the hair before, during, and/or after chemical treatment of the hair, such as before, during, and/or after application a bleaching process, a coloring process, a relaxing process, a permanent waving process, or a straightening process.

A further exemplary and non-limiting method according to the disclosure comprises applying a first composition comprising at least one amino acid chosen from glycine, at least one cationic polymer chosen from homopolymers of diallyl dimethyl ammonium chloride (polyquaternium-6), and optionally at least one solvent chosen from water, to the hair, before, at substantially the same time as, and/or after applying a second composition comprising at least one carboxylic acid chosen from maleic acid and salts thereof, at least one amine chosen from monoethanolamine, and optionally at least one solvent to the hair, before, during, and/or after chemical treatment of the hair, such as before, during, and/or after application a bleaching process, a coloring process, a relaxing process, a permanent waving process, or a straightening process.

By way of non-limiting example, methods according to the disclosure may comprise applying compositions according to the disclosure on to the hair as a pre-treatment composition before the hair is chemically treated (e.g. lightened, colored, and/or relaxed). The pre-treatment composition may optionally be left on the hair or rinsed or washed out.

As a further non-limiting example, methods according to the disclosure may comprise applying compositions according to the disclosure on to the hair as a post-treatment composition after the hair has been chemically treated (e.g. lightened, colored, and/or relaxed). The post-treatment composition may optionally be left on the hair or rinsed or washed out.

In various embodiments, upon application of a composition according to the disclosure to the hair, and after an optional resting time (leave-on time) on the hair fibers, for example, ranging from about 1 to about 60 minutes, such as from about 5 to about 45 minutes, or such as from about 5 to about 20 minutes, or such as from about 10 to about 20 minutes, or such as of about 20 minutes, the keratinous substrates are rinsed, optionally washed with shampoo, rinsed again, optionally washed with a hair conditioning composition, and rinsed again, then dried. The shampoo and hair conditioning composition can be any conventional hair shampoo and/or conditioner products.

The temperature at which compositions are applied to the hair according to various methods of the disclosure may, for example, range from room temperature and 80° C., such as between room temperature and 60° C. The hair fibers may optionally be heated before, while, and/or after a composition according to the disclosure is in contact therewith, for example may be heated at a temperature ranging up to 200° C. The hair fibers may be heated by any conventional means, such as, for example, by applying heat via a heat source such as a flat iron, curling iron, hair dryer, and the like.

It has been discovered that the application of compositions according to the disclosure before, during, and/or after chemical treatment of keratinous fibers such as the hair results in satisfactory chemical treatment of the fibers, while providing one or more of shine, conditioning, strength, protection, and/or a healthy appearance to the fibers so as to avoid, minimize, and/or repair damage to the fibers in at least some embodiments. It is to be understood, however, that any degree of shine, conditioning, fiber strength, protection, and/or a healthy appearance may be imparted to the fibers, without limitation. In addition, it is intended that embodiments that do not impart one or more of shine, conditioning, fiber strength, protection, and/or a healthy appearance to the hair fiber are also within the scope of the disclosure.

### Kits

Further embodiments of the disclosure relate to kits for chemical treatment of keratinous fibers, such as the hair. Various embodiments of kits for altering the color of hair comprise:

a first compartment comprising a composition comprising:

at least one amino acid;

at least one carboxylic acid;

at least one amine; and

at least one cationic polymer;

a second compartment comprising at least one oxidizing agent; and

optionally, a third compartment comprising at least one colorant or bleaching agent.

Various embodiments of kits for altering the shape of hair comprise:

a first compartment comprising a composition comprising:

at least one amino acid;

at least one carboxylic acid;

at least one amine; and

at least one cationic polymer;

a second compartment comprising at least one agent for altering the shape of the hair, such as an agent for permanent waving, relaxing, or straightening the hair; and

optionally, a third compartment comprising at least one neutralizing agent.

One exemplary and non-limiting example of a kit for altering the color of hair comprises:

a first compartment comprising a composition comprising:

at least one amino acid chosen from glycine;

at least one carboxylic acid chosen from maleic acid and salts thereof;

at least one amine chosen from monoethanolamine; and

at least one cationic polymer chosen from homopolymers of diallyl dimethyl ammonium chloride (polyquaternium-6);

a second compartment comprising at least one oxidizing agent; and

optionally, a third compartment comprising at least one colorant or bleaching agent.

One exemplary and non-limiting example of a kit for altering the shape of hair comprises:

a first compartment comprising a composition comprising:

at least one amino acid chosen from glycine;

at least one carboxylic acid chosen from maleic acid and salts thereof;

at least one amine chosen from monoethanolamine; and

at least one cationic polymer chosen from homopolymers of diallyl dimethyl ammonium chloride (polyquaternium-6);

a second compartment comprising at least one agent for altering the shape of the hair, such as an agent for permanent waving, relaxing, or straightening the hair; and

optionally, a third compartment comprising at least one neutralizing agent.

Further exemplary embodiments of kits for altering the color of hair comprise:

a first compartment comprising a composition comprising:

at least one amino acid; and

at least one cationic polymer;

a second compartment comprising a composition comprising:

at least one carboxylic acid; and

at least one amine;

a third compartment comprising at least one oxidizing agent; and

optionally, a fourth compartment comprising at least one colorant or bleaching agent.

Further exemplary embodiments of kits for altering the shape of hair comprise:

a first compartment comprising a composition comprising:

at least one amino acid; and

at least one cationic polymer;

a second compartment comprising a composition comprising:

at least one carboxylic acid; and

at least one amine;

a third compartment comprising at least one agent for altering the shape of the hair, such as an agent for permanent waving, relaxing, or straightening the hair; and

optionally, a fourth compartment comprising at least one neutralizing agent.

An exemplary and non-limiting example of a kit for altering the color of hair comprises:

a first compartment comprising a composition comprising:

at least one amino acid chosen from glycine; and

at least one cationic polymer chosen from homopolymers of diallyl dimethyl ammonium chloride (polyquaternium-6);

a second compartment comprising a composition comprising:

at least one carboxylic acid chosen from maleic acid and salts thereof; and

at least one amine chosen from monoethanolamine;

a third compartment comprising at least one oxidizing agent; and

optionally, a fourth compartment comprising at least one colorant or bleaching agent.

An exemplary and non-limiting example of a kit for altering the shape of hair comprises:

a first compartment comprising a composition comprising:

at least one amino acid chosen from glycine; and

at least one cationic polymer chosen from homopolymers of diallyl dimethyl ammonium chloride (polyquaternium-6);

a second compartment comprising a composition comprising:

at least one carboxylic acid chosen from maleic acid and salts thereof; and

at least one amine chosen from monoethanolamine;

a third compartment comprising at least one agent for altering the shape of the hair, such as an agent for permanent waving, relaxing, or straightening the hair; and

optionally, a fourth compartment comprising at least one neutralizing agent.

It to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

Unless otherwise expressly stated, it is in no way intended that any method set forth herein be construed as requiring that its steps be performed in a specific order. Accordingly, where a method claim does not actually recite that an order is to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

All patents and publications disclosed herein are expressly incorporated by reference in their entireties.

### EXAMPLES

The following Examples are provided for illustrative purposes only, and are not intended to be limiting.

### Example 1

The ingredient amounts in the compositions described below are expressed in % by weight, based on the total weight of the cosmetic composition/formula, in Table 1-4 below.

Post Treatment 1 (Comparative):

A commercially available post treatment formulation comprising maleic acid and a diamine was applied to the hair after application of a guanidine relaxer. The listed active agent disclosed was a salt of maleic acid, bis-aminopropyl diglycol dimaleate. The hair was subsequently rinsed and evaluated using the methods described below.

Post Treatment 2 (Comparative):

Formula A of Table 1 and Formula B of Table 2 were applied sequentially to the hair after the application of a guanidine relaxer.

TABLE 1

| | Formula A - % by |
|---|---|
| INCI ingredient | weight |
| Maleic acid | 10.7 |
| Ethanolamine | 5.4 |
| Water | 83.9 |
| Total | 100 |

TABLE 2

| | Formula B - % by |
|---|---|
| INCI ingredient | weight |
| Water | 86.41% |
| Emulsifying Agents | 4.19% |
| Humectants | 3.5% |
| Conditioning Agents | 2.03% |
| Maleic Acid | 1.9% |
| Preseravatives | 0.7 |
| Emollients | 0.16% |
| Film Former | 0.012% |
| Ethanolamine | 0.6% |
| Thickeners | 0.5% |

Post Treatment 3 (Inventive):

Formula C of Table 3 was applied to the hair after treatment with a guanidine relaxer.

TABLE 3

| | Formula C - % by |
|---|---|
| INCI ingredient | weight |
| Maleic acid | 10.701 |
| Ethanolamine | 5.4 |
| Polyquaternium-6 | 0.4 |
| Glycine | 2.5 |
| Citric acid | 0.1 |
| Water | 80.899 |
| Total: | 100 |

Hair swatches were subjected to a hair relaxing treatment. Subsequently, the hair was rinsed and subjected to a post-treatment as defined above. The fiber integrity of the hair was evaluated using a Dia-stron7 Miniature Tensile Tester (MTT). In addition, the technical shine was evaluated using the Reich-Robbins method. The results are shown in Table 4.

TABLE 4

| | Elastic Modulus | Break Stress | Shine |
|---|---|---|---|
| Post Treatment | (MPa) | (MPa) | (Reich-Robbins) |
| None | 789.22 | 109.31 | 210.95 |
| 1 (Comparative) | 712.07 | 109.8 | 293.39 |
| 2 (Comparative) | 757.86 | 118.82 | 259.38 |
| 3 (Inventive) | 1032.74 | 127.99 | 318.57 |

As shown in Table 4, the elastic modulus, break stress, and shine were higher for post treatment 3 than for the comparative treatments, demonstrating improved resistance to breakage and increased shine in hair treated with post treatment 3.

### Example 2

Formula D (inventive) and Formula E (comparative) were prepared as follows, for evaluation with hair relaxing compositions.

TABLE 5

| INCI ingredient | Formula D - % by weight |
|---|---|
| fragrance | 0.3 |
| trideceth-2 carboxamide MEA | 2.43 |
| maleic acid | 1.899 |
| caprylyl glycol | 0.5 |
| cetyl hydroxyethylcellulose | 0.2 |
| sodium stannate | 0.02 |
| ethanolamine | 0.8 |
| polyquaternium-6 | 0.4 |
| glycine | 2.5 |
| glycerin | 1.75 |
| edta | 0.28 |
| alcohol | 0.015 |
| laureth-12 | 1.5 |
| laureth-2 | 4 |
| oleth-30 | 1 |
| sodium cetearyl sulfate | 0.72 |
| tetrasodium pyrophosphate | 0.01 |
| water aqua | 79.803 |
| sodium sulfate | 0.015 |
| sodium sulfite | 0.01 |
| citric acid | 0 |
| peg-4 rapeseedamide | 1.848 |
| Total: | 100 |

TABLE 6

| INCI ingredient | Formula E - % by weight |
|---|---|
| fragrance | 0.15 |
| trideceth-2 carboxamide mea | 2.43 |
| caprylyl glycol | 0.5 |
| cetyl hydroxyethylcellulose | 0.2 |
| sodium stannate | 0.02 |
| pentasodium pentetate | 0.032 |
| ethanolamine | 0 |
| polyquaternium-6 | 0.4 |
| glycine | 2.5 |
| glycerin | 1.75 |
| edta | 0.2 |
| cetearyl alcohol | 0.015 |
| laureth-12 | 1.5 |
| laureth-2 | 4 |
| oleth-30 | 1 |
| sodium cetearyl sulfate | 0.72 |
| tetrasodium pyrophosphate | 0.01 |
| water aqua | 82.7 |
| sodium sulfate | 0.015 |
| sodium sulfite | 0.01 |
| citric acid | 0 |
| peg-4 rapeseedamide | 1.8480 |
| Total: | 100 |

Hair relaxing compositions were prepared as shown in Table 7 and applied to the hair. The hair was subsequently rinsed, and the pH of the hair after treatment was evaluated.

TABLE 7
HAIR RELAXING COMPOSITIONS

| | | pH after |
|---|---|---|
| Formula | Composition | Treatment |
| 1. Comparative | Relaxer + Formula A | 5.91 |
| 2. Comparative | Relaxer + Formula A + Formula B | 5.16 |
| 3. Comparative | Relaxer + Formula E | 6.24 |
| 4. Inventive | Relaxer + Formula C | 4.95 |
| 5. Inventive | Relaxer + Formula C + Formula B | 4.20 |
| 6. Inventive | Relaxer + Formula C + Formula D | 4.38 |
| 7. Comparative | Relaxer Alone + Neutralizing Conditioner + | 9.13 |
| | Shampoo | |

As can be seen from Table 7, hair that is treated with hair relaxing compositions comprising at least one amino acid, at least one carboxylic acid, at least one amine, and at least one cationic polymer (formulas 4-6) had lower pH values, demonstrating more effective neutralization in conjunction with the relaxing process.

What is claimed is:

1. A composition comprising:
at least one amino acid;
at least one carboxylic acid;
at least one amine; and
at least one cationic polymer.

2. The composition of claim 1, wherein the at least one amino acid is chosen from glycine, alanine, serine, proline, or mixtures thereof.

3. The composition of claim 1, wherein the at least one carboxylic acid is chosen from oxalic acid, malonic acid, malic acid, glutaric acid, citraconic acid, citric acid, maleic acid, glycolic acid, succinic acid, adipic acid, tartaric acid, fumaric acid, adipic acid, sebacic acid, benzoic acid, or salts thereof.

4. The composition of claim 1, wherein the at least one amine is chosen from monoethanolamine, diethanolamine, triethanolamine, or mixtures thereof.

5. The composition of claim 1, wherein the at least one cationic polymer is chosen from polymeric quaternary ammonium salts.

6. The composition of claim 5, wherein the polymeric quaternary ammonium salt is chosen from cationic homopolymers of diallyl dimethyl ammonium chloride.

7. The composition of claim 1, wherein the composition further comprises at least one solvent.

8. The composition of claim 1, further comprising at least one agent for chemically treating the hair.

9. The composition of claim 8, wherein the agent for chemically treating the hair is a hair shaping agent chosen from sodium hydroxide, potassium hydroxide, lithium hydroxide, calcium hydroxide, magnesium hydroxide, barium hydroxide, strontium hydroxide, guanidine hydroxide, cysteine, cysteamine, N-substituted cysteamines, alkyl substituted mercaptoacetamides, dimercaptoadipic acid, thioglycerol, thiolactic acid, thioglycolic acid, monothioglycolic acid esters such as diol esters of thioglycolic acid, glyceryl monothioglycolate, thiocholine, thiols, guanidine, sulfites, bisulfites, lithium carbonate, sodium carbonate, potassium carbonate, guanidine carbonate, or salts or derivatives thereof.

10. A composition comprising:
at least one amino acid chosen from glycine;
at least one carboxylic acid chosen from maleic acid and salts thereof;
at least one amine chosen from monoethanolamine; and
at least one cationic polymer chosen from homopolymers of diallyl dimethyl ammonium chloride (polyquaternium-6).

11. A method for treating the hair comprising:
applying a chemical treatment composition to the hair;
wherein the chemical treatment composition is chosen from compositions for dyeing, lifting, bleaching, permanent waving, or relaxing treatment compositions; and
applying a composition to the hair before, during, and/or after the chemical treatment composition, wherein said composition comprises:
at least one amino acid;
at least one carboxylic acid;
at least one amine;
at least one cationic polymer; and
optionally at least one solvent.

12. The method of claim 11, wherein the at least one amino acid is chosen from glycine, alanine, serine, proline, or mixtures thereof.

13. The method of claim 11, wherein the at least one amine is chosen from monoethanolamine, diethanolamine, triethanolamine, or mixtures thereof.

14. The method of claim 11, wherein the at least one cationic polymer is chosen from polymeric quaternary ammonium salts.

15. The method of claim 14, wherein the at least one cationic polymer is chosen from cationic homopolymers of diallyl dimethyl ammonium chloride.

16. The method of claim 11, wherein the at least one carboxylic acid is chosen from oxalic acid, malonic acid, malic acid, glutaric acid, citraconic acid, citric acid, maleic acid, glycolic acid, succinic acid, adipic acid, tartaric acid, fumaric acid, adipic acid, sebacic acid, benzoic acid, or salts thereof.

17. The method of claim 11, wherein the pH of the hair is less than about 5 after applying the composition to the hair.

18. The method of claim 11, wherein the step of applying a chemical treatment composition to the hair comprises applying a composition comprising guanidine to the hair.

19. A method for treating the hair comprising:
applying a chemical treatment composition to the hair;
wherein the chemical treatment composition is chosen from compositions for dyeing, lifting, bleaching, permanent waving, or relaxing treatment compositions; and
applying a composition to the hair before, during, and/or after the chemical treatment composition, wherein said composition comprises:
at least one amino acid chosen from glycine;
at least one carboxylic acid chosen from maleic acid and salts thereof;
at least one amine chosen from monoethanolamine; and
at least one cationic polymer chosen from homopolymers of diallyl dimethyl ammonium chloride (polyquatemium-6).

20. A method for treating the hair comprising:
applying a composition according to claim 1 to the hair.

21. A method for treating the hair comprising:
applying a composition according to claim 10 to the hair.

22. A kit comprising:
a first compartment comprising a composition according to claim 1; and
a second compartment comprising a chemical treatment composition;
wherein the chemical treatment composition is chosen from compositions for dyeing the hair, lifting the color of the hair, bleaching the hair, permanent waving the hair, straightening the hair, or relaxing the hair.

23. A kit comprising:
a first compartment comprising a composition according to claim 10; and
a second compartment comprising a chemical treatment composition;
wherein the chemical treatment composition is chosen from compositions for dyeing the hair, lifting the color of the hair, bleaching the hair, permanent waving the hair, straightening the hair, or relaxing the hair.
